(19) United States
(12) Patent Application Publication
(10) Pub. No.: US 2008/0215124 A1
(43) Pub. Date: Sep. 4, 2008

(54) Apparatus for Cosmetic Skin Rejuvenation Treatment

(75) Inventors: Giovanna Wagenaar Cacciola, Eindhoven (NL); Vincent Fischer, Eindhoven (NL); Marco Haverlag, Eindhoven (NL); Robert John De Jonge, Turnhout (BE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/917,204

(22) PCT Filed: Jun. 13, 2006
(86) PCT No.: PCT/IB2006/051882

§ 371 Date: Dec. 12, 2007

(30) Foreign Application Priority Data
Jun. 14, 2005 (EP) 05105195.1

(51) Int. Cl. A61N 5/06; A61B 18/18

(52) U.S. Cl. 607/90; 607/88; 606/3; 606/9; 606/2

(57) ABSTRACT

The present invention relates to an apparatus for cosmetic skin rejuvenation treatment, comprising a continuous wave light source accommodating a high-intensity discharge lamp (104; 204; 304; 604) which has at least one predominant spectral peak between 600 and 700 nm.

### FIELD OF THE INVENTION

The present invention relates to the field of phototherapy of the skin.

### BACKGROUND OF THE INVENTION

Skin rejuvenation treatments are performed nowadays in the professional sector with lasers or IPL (Intense Pulsed Light), which are either ablative, i.e. they burn a layer of the skin, starting a wound healing process which leads to formation of new collagen so that the skin looks younger, or which are non ablative, stimulating the collagen production without burning the skin (in this case the treatment is called just “wrinkles reduction”). For acne treatment, lasers or IPL give enough energy to effectively kill de acne bacteria or destroy the sebaceous glands.

There is a recent trend in the professional market to go to lower energy therapies, with a continuous light source like LEDs, which could be use alone or in synergy with an IPL or laser treatments. But, especially for the consumer market a low energy light source is mandatory, for safety reasons. These low energy light sources relate to LEDs or TL's and both lamp types seem to give a lower power than professional devices, which means they are less effective than professional devices. For these consumer applications, in order to reach a comparable (medical) efficacy as professional devices, TL or a low number of LEDs, such as in the devices now available in the consumer market (see FIGS. 1 and 2) are not enough.

To reach the right power needed to make the treatment effective, a lot of LEDs must be used (for instance 1600 LEDs for GentleWaves, a device sold by Light Bioscience for wrinkles reduction). This makes the cost for such a device (>10̂4 $) too high for a consumer application.

From the patent literature various apparatuses and methods for light therapy are known. For example, U.S. Pat. No. 5,549,660 shows a method of treating acne. The acne treatment is performed by illumination using light-emitting diodes in a continuous-wave mode of operation to generate continuous wave light radiation in a narrow bandwidth. The continuous wave light radiation is concentrated and projected onto an acne-affected dermal zone for biostimulative treatment.

In addition, high-intensity visible light phototherapy for acne was described by Meffert et al. (Dermatol-Monatsschr. 1990; 176(10): 597-603), and in U.S. Pat. Nos. 6,676,655 and 6,626,932.

### SUMMARY OF THE INVENTION

According to the present invention, an apparatus for cosmetic skin rejuvenation treatment comprises a continuous wave light source accommodating a high-intensity discharge lamp, the high-intensity discharge lamp having at least one predominant spectral peak between 550 and 700 nm, preferably between 650 and 700 nm.

It has been found that treatment of the human skin with radiation of a wavelength between 550 and 700 nm can reduce wrinkles. Use of a high-intensity discharge lamp for generating this radiation is cost-efficient. The present invention thus facilitates the design of a relatively inexpensive consumer device for skin rejuvenation treatment.

In accordance with an embodiment of the invention, the high-intensity discharge lamp has a salt filling. When the high-intensity discharge lamp is switched on, the salt evaporates. The evaporated salt shifts the wavelength of the radiation emitted by the high-intensity discharge lamp into the 550 to 700 nm range that is required for skin rejuvenation treatment. Conventional Continuous Wave light sources are designed to generate white light and thus emit a continuous full spectrum. Application of these light sources for skin rejuvenation, renders these light sources to be less efficient than the light source according to the invention as the emitted spectrum of the conventional light sources must be filtered to get the right wavelengths.

In accordance with an embodiment of the invention, the salt filling comprises a halide, such as lithium halide, sodium halide, cesium halide, and/or calcium halide, in particular lithium iodide (LiI), sodium iodide (NaI), cesium iodide (CsI) and/or calcium iodide (CaI₂) and/or respective bromides, fluorides, and/or chlorides. In addition, the salt may contain other metal halides to stabilize the arc of the discharge lamp.

Preferably, a lithium halide lamp having a spectral peak between 550 and 700 nm, preferably between 650 and 700 nm, is used for wrinkle reduction. For example, the treatment is performed with a fluence of >10 J/cm² and a treatment time of over 100 seconds.

In accordance with an embodiment of the invention, the high-intensity discharge lamp has a gas filling containing argon, xenon, krypton, and/or neon.

The high-intensity discharge lamp may or may not have a mercury filling. In accordance with an embodiment of the invention, the plasma chamber of the high-intensity discharge lamp has a quartz glass wall. The quartz glass has a silicon dioxide (SiO₂) content of at least 95% by weight.

In accordance with an embodiment of the invention, the plasma chamber of the high-intensity discharge lamp has a ceramic wall, such as a PCA wall (Poly Cristalline Alumina; AL₂O₃ in polycrystalline form).

In accordance with an embodiment of the invention, the continuous wave light source can operate with an alternative high-intensity discharge lamp for treatment of a skin disorder, such as acne. The alternative high-intensity discharge lamp has at least one spectral peak between 390 and 430 nm. Such a radiation spectrum is obtained e.g. by means of a halide salt filling, such as a gallium iodide (GaI₃), aluminum iodide (AlI₃) and/or indium iodide (InI₃) salt filling. Again, the gas filling may be argon, xenon, krypton and/or neon with or without mercury.

In accordance with an embodiment of the invention, the continuous wave light source has means for (releasably) receiving the high-intensity discharge lamp or the alternative high-intensity discharge lamp. For example, for wrinkle treatment, the high-intensity discharge lamp having a spectral peak between 550 and 700 nm is inserted into the light source, whereas for treatment of a skin disorder, such as acne, the high-intensity discharge lamp is replaced with the alternative high-intensity discharge lamp that has at least one predominant peak in the 390 to 430 nm range.

In accordance with an embodiment of the invention, the high-intensity discharge lamp and the alternative high-intensity discharge lamp are mounted on a rotational spindle for pivoting one or the other lamp into a position suitable for use.

In accordance with still another embodiment of the present invention, both the high-intensity discharge lamp and the alternative high-intensity discharge lamp are permanently mounted within the light source, such as close to the focal point of a reflector. The apparatus has a control element, such as a control button to enable a user to select a first or a second mode of operation.

In the first mode of operation, at least the high-intensity discharge lamp with the peak in the 550 to 700 nm range is switched on for skin rejuvenation treatment, whereas in the second mode of operation, at least the alternative high-intensity discharge lamp is switched on for treatment of a skin disorder, such as acne.

Rather than switching the lamps on or off in dependence upon the mode, it is also possible to vary the respective radiation intensities. For example, in the first mode of operation, the high-intensity discharge lamp having the peak in the 550 to 700 nm range is operated to produce high-intensity radiation, whereas the alternative high-intensity discharge lamp is operated at a low radiation intensity level. In the second mode of operation, the alternative high-intensity discharge lamp having a spectral peak in the 390 to 430 nm range is operated with high radiation intensity, whereas the high-intensity discharge lamp that has the spectral peak in the 550 to 700 nm range is operated at a lower radiation intensity level.

In accordance with an embodiment of the invention, both skin rejuvenation treatment, such as anti-wrinkle treatment, and treatment of a skin disorder, such as acne, can be accomplished with a single lamp that has a filling comprising at least two different salts, such as lithium iodide and gallium iodide, resulting in respective spectral peaks.

In accordance with an embodiment of the invention, the apparatus for skin rejuvenation treatment is portable. The apparatus is preferably a table-top unit. This is particularly advantageous for consumer devices because an expensive mechanical support, such as a tripod, for holding and positioning the apparatus can be dispensed with.

### BRIEF DESCRIPTION OF THE DRAWINGS

Preferred embodiments of the present invention will be described hereinafter in greater detail with reference to the drawings, in which:

FIG. 1 is a schematic cross-sectional view of a first embodiment of an apparatus of the invention,

FIG. 2 is a schematic cross-sectional view of a second embodiment of an apparatus of the invention,

FIG. 3 shows the embodiment of FIG. 2 after pivoting of the lamps,

FIG. 4 is a schematic cross-sectional view of a third embodiment of an apparatus of the invention,

FIG. 5 is a cross-sectional view of a quartz high-intensity discharge lamp,

FIG. 6 is a cross-sectional view of a PCA high-intensity discharge lamp,

FIG. 7 shows a spectrum of a high-intensity discharge lamp with a lithium iodide (LiI) salt filling,

FIG. 8 shows a spectrum of a high-intensity discharge lamp with a gallium iodide (Gal) salt filling,

FIG. 9 shows a spectrum of a high-intensity discharge lamp with both a lithium iodide (LiI) and a gallium iodide (GaI3) salt filling,

FIG. 10 shows a table-top embodiment of an apparatus of the invention in a position suitable for use,

FIG. 11 shows an embodiment of an apparatus of the invention to be hung on a wall in a position suitable for use.

### DESCRIPTION OF EMBODIMENTS

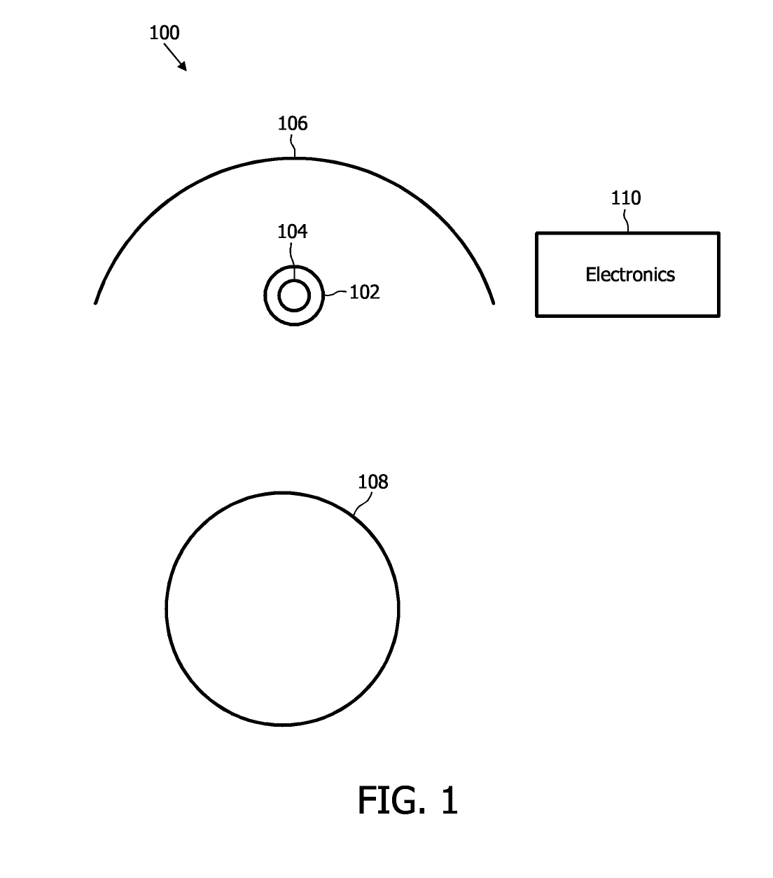

FIG. 1 shows an apparatus 100 for cosmetic skin rejuvenation treatment. The apparatus has a continuous wave light source comprising a socket 102 for receiving a high-intensity discharge lamp 104. The high-intensity discharge lamp 104 is positioned in a focal area of a reflector 106. The reflector 106 concentrates and directs the light emitted from the high-intensity discharge lamp 104 towards a person 108 who is sitting in front of the apparatus 100. The operation of the apparatus 100 is controlled by an electronics module 110 which has also a current-limiting function for operation of the high-intensity discharge lamp 104. This current-limiting function can be implemented as an inductor, a transformer ballast or, alternatively, by using an electronic lamp driver module operating with a square-wave current profile or a high-frequency sinusoidal current profile.

The high-intensity discharge lamp 104 has a predominant spectral peak between 550 and 700 nm.

The skin of the person 108 is preferably exposed to a light dose of at least 10 J/cm² or more. For example, the high-intensity discharge lamp 104 has a radiation efficiency of more than 16% and a power consumption above 200 W. Such a set-up provides an efficient skin rejuvenation treatment, such as wrinkle treatment, for the person 108.

The high-intensity discharge lamp 104 can be removed from the socket 102 and an alternative high-intensity discharge lamp having a different radiation spectrum can be plugged into the socket for another application.

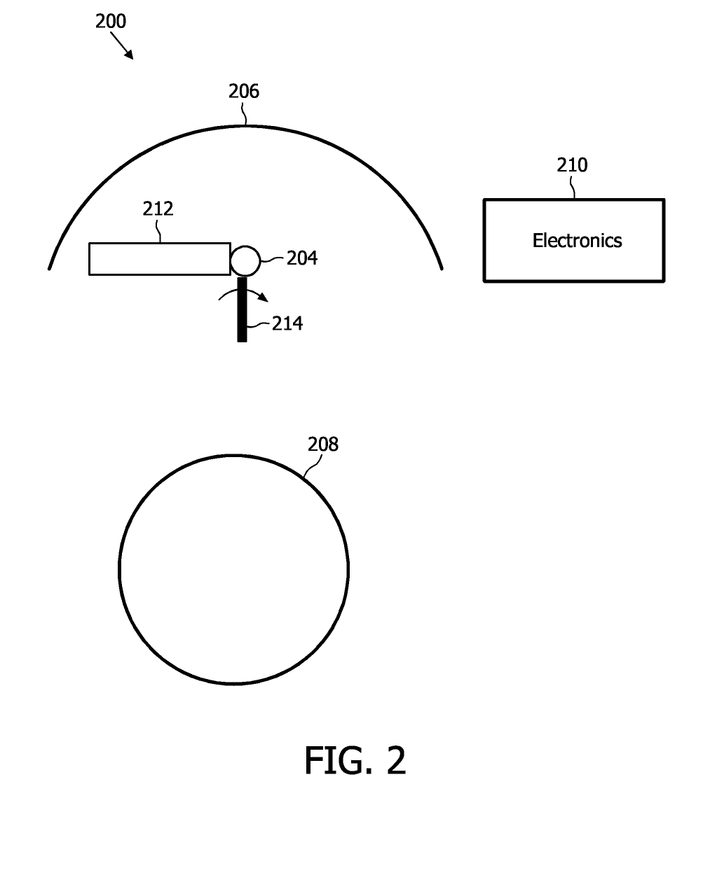

FIG. 2 shows an alternative embodiment. In the following Figures, the same reference numerals will be used to denote identical elements.

The apparatus 200 is a dual-mode device which can be used for skin rejuvenation, such as wrinkle treatment, as well as for treatment of skin disorders, such as acne. For this purpose, the apparatus 200 has an additional high-intensity discharge lamp 212 which, together with the high-intensity discharge lamp 204, is mounted on a rotational spindle 214 of the apparatus 200. The high-intensity discharge lamp 204 has at least one predominant peak in the 550 to 700 nm range and is used for skin rejuvenation treatment, whereas the high-intensity discharge lamp 212 has at least one predominant peak in the 390 to 430 nm range for treatment of skin disorders, such as acne.

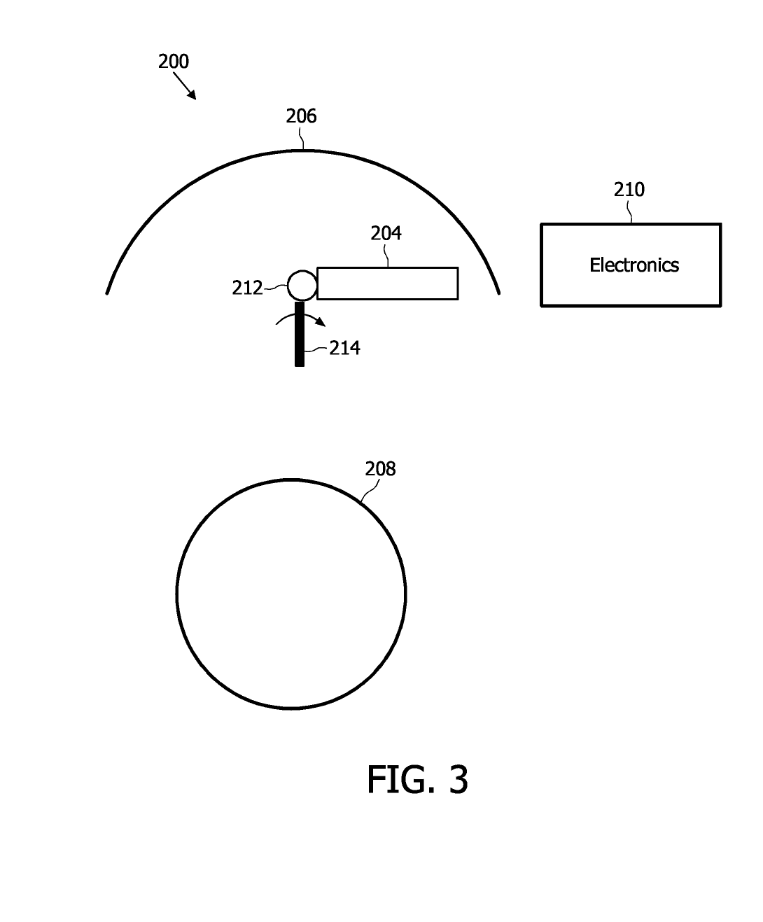

As is shown in FIG. 2, the high-intensity discharge lamp 204 and the high-intensity discharge lamp 212 are arranged perpendicularly. When the high-intensity discharge lamp 204 is in a vertical position, as shown in FIG. 2, the apparatus 200 can be operated in a first mode for skin rejuvenation. By pivoting the high-intensity discharge lamp 204 and the high-intensity discharge lamp 212 around the rotational spindle 214, the high-intensity discharge lamp 204 is brought to a horizontal position and the high-intensity discharge lamp 212 is brought to a vertical position as shown in FIG. 3. After pivoting the high-intensity discharge lamp 212 into its vertical position, the apparatus 200 can be used in its second mode of operation for treatment of a skin disorder. The electronics module 210 serves for powering either the high-intensity discharge lamp 204 or the high-intensity discharge lamp 212, depending on the selected mode of operation of the apparatus 200.

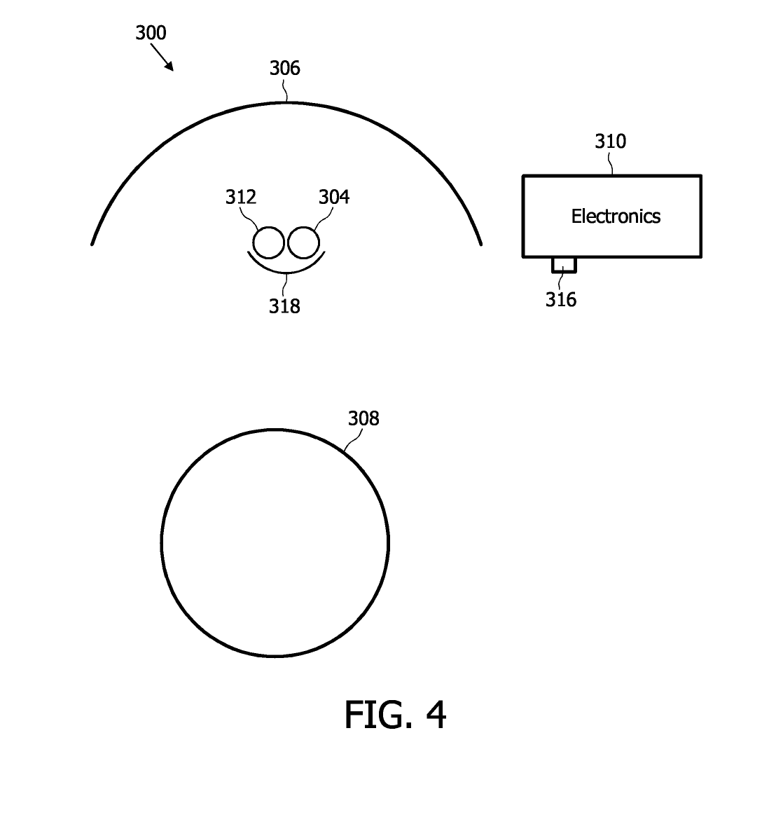

FIG. 4 shows still another embodiment of an apparatus 300. The apparatus 300 has a high-intensity discharge lamp 304 with at least one predominant peak in the 550 to 700 nm range, as well as a high-intensity discharge lamp 312 with at least one predominant peak in the 390 to 430 nm range. Both high-intensity discharge lamps 304 and 312 are fixedly mounted within the apparatus 300.

The electronics module 310 is coupled to a mode selection button 316 to enable the person 308 to select one of the modes of operation of the apparatus 300. For example, the mode selection button 316 has two switch positions, one for selecting the first mode of operation for skin rejuvenation treatment and the other for selecting the second mode of operation for skin disorder treatment.

In the first mode of operation, only the high-intensity discharge lamp 304 is switched on, whereas only the high-intensity discharge lamp 312 is switched on in the second mode of operation. Alternatively, the respective intensities of the high-intensity discharge lamps 304, 312 are varied in dependence upon the selected mode of operation.

The apparatus 300 preferably has a shield 318 which reflects light emitted from the high-intensity discharge lamp 304 and/or 312 onto the reflector 306 in order to prevent glare hindering the person 308. The shield 318 may be a separate component or may be integrated in each high-intensity discharge lamp 304, 312. Such an antiglare shield precludes direct irradiation from the lamp (the light that reaches the face, or accidentally the eye, is only reflected). The shield may be a metal screen in front of the lamp, or a reflective layer in the front of the outer bulb of the lamp.

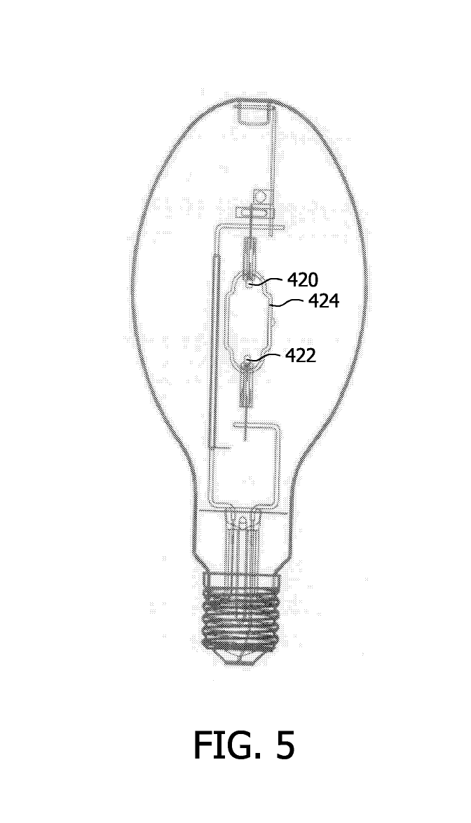

FIG. 5 is a cross-sectional view of an embodiment of a high-intensity discharge lamp. The high-intensity discharge lamp has electrodes 420, 422 with a plasma chamber 424. The electrodes 420, 422 are spaced apart by approximately 10 to 50 mm. The plasma chamber 424 is filled with argon, xenon, krypton and/or neon with or without mercury. In addition, the plasma chamber 424 has a halide salt filling, such as lithium iodide, sodium iodide, cesium iodide, calcium iodide, gallium iodide, aluminum iodide, and/or an indium iodide salt filling.

The wall of the plasma chamber 424 is made of quartz glass.

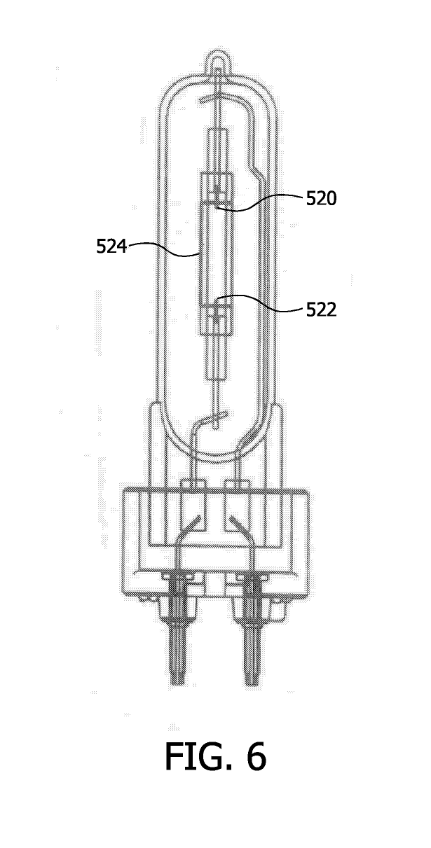

FIG. 6 is a cross-sectional view of an alternative embodiment of a high-intensity discharge lamp. In the embodiment considered here, the wall of the plasma chamber 524 is made of PCA.

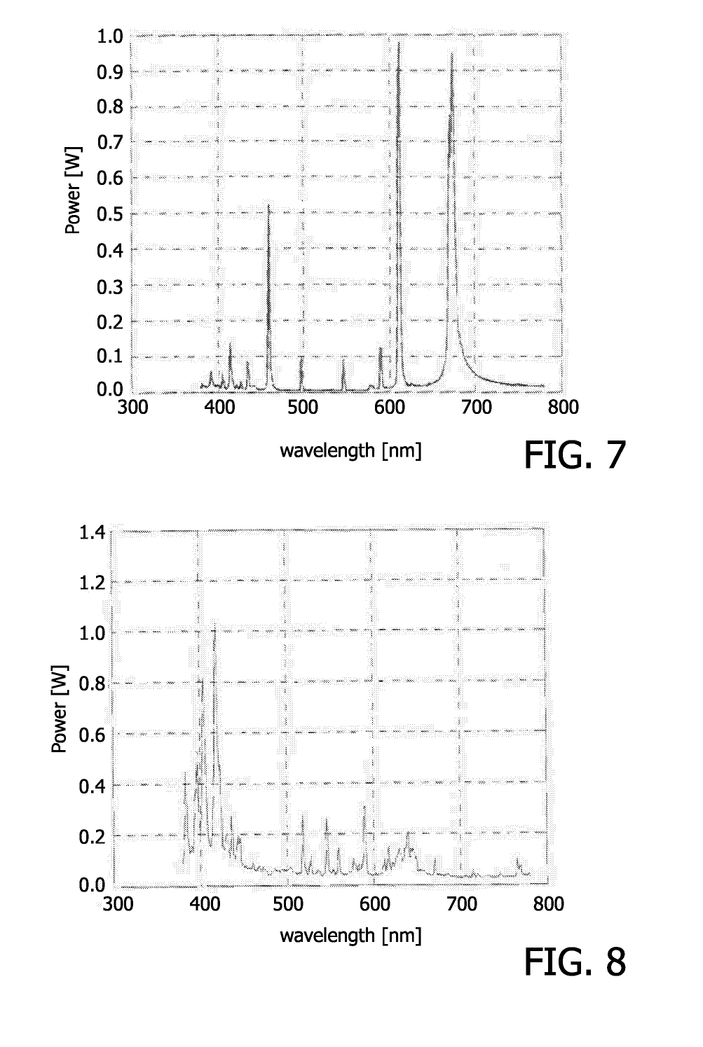

FIG. 7 shows a spectrum of a high-intensity discharge lamp with a lithium iodide salt filling. The high-intensity discharge lamp has two predominant peaks in the 550 to 700 nm range and can thus be used for skin rejuvenation treatment.

FIG. 8 shows a spectrum of a high-intensity discharge lamp with a gallium iodide salt filling. The lamp has predominant peaks in the 390 to 430 nm range, so that it can be used for treatment of skin disorders, such as acne.

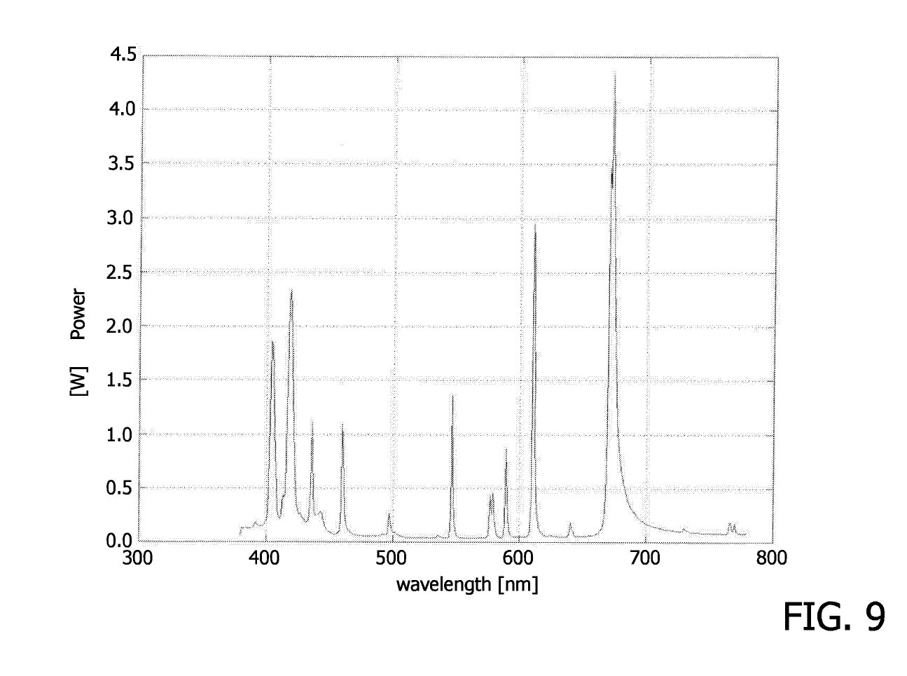

FIG. 9 shows a spectrum of a high-intensity discharge lamp with both a lithium iodide and a gallium iodide salt filling. The high-intensity discharge lamp has predominant peaks in the 390 to 430 nm range and in the 550 to 700 nm ranges and can thus be used very favorably for skin disorder treatment as well as for skin rejuvenation treatment.

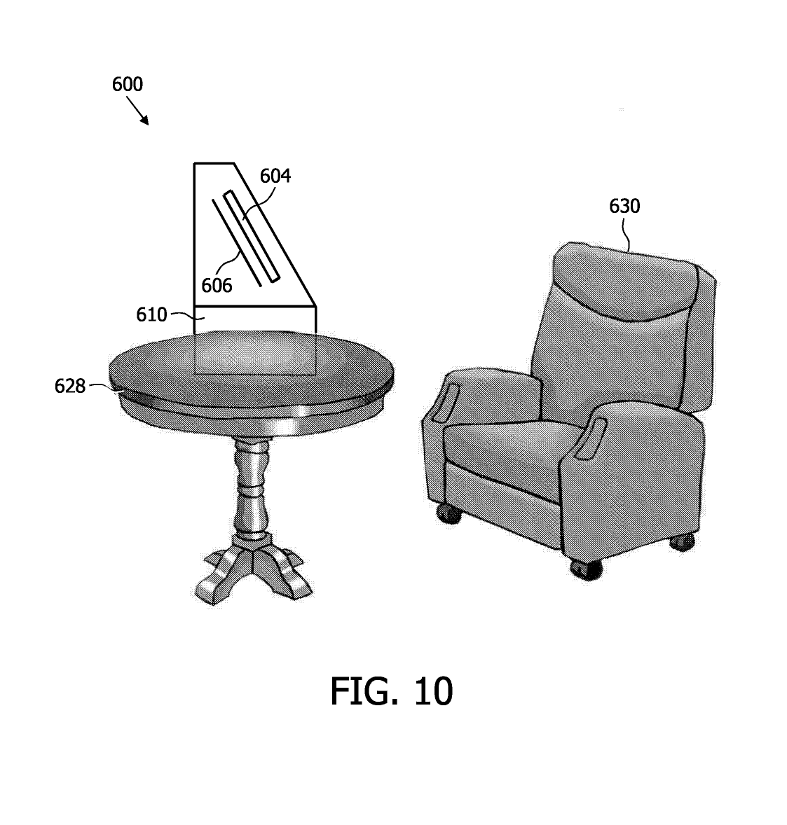

FIG. 10 shows an embodiment of an apparatus 600 for home use. The apparatus 600 is portable and can be used as a table-top unit. The housing 626 of the apparatus 600 accommodates the electronics module 610, at least one high-intensity discharge lamp 604 and a reflector 606. The reflector 606 and the at least one high-intensity discharge lamp 604 are mounted in an inclined position at an angle of inclination of e.g. 10 to 40°, preferably 30°, with respect to the vertical direction. When the apparatus 600 is put on a table 628, a person can sit down on armchair 630 so as to be conveniently exposed to the radiation emitted from the apparatus 600.

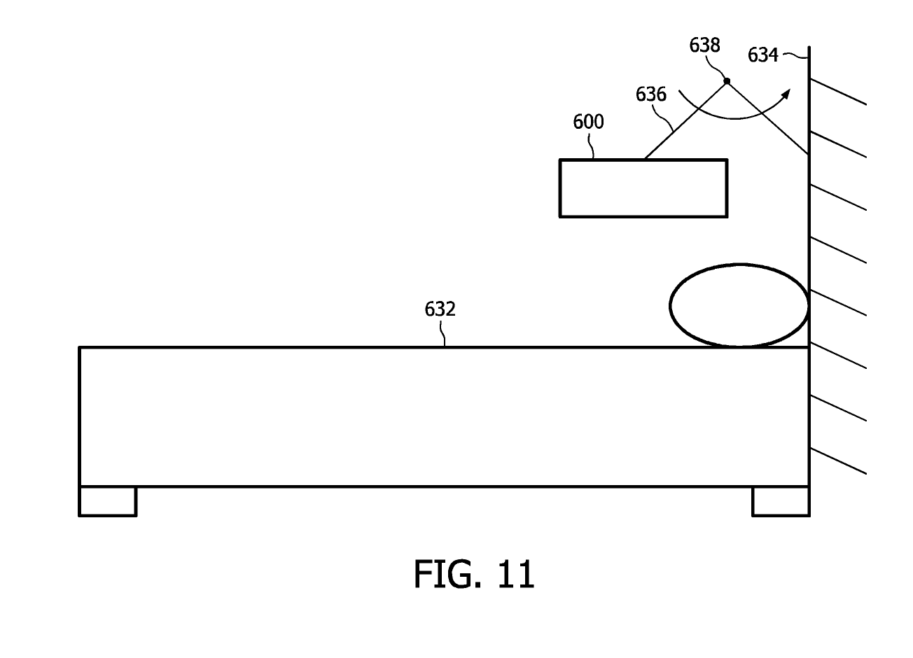

The housing of the apparatus 600 is preferably designed to be hung on a wall 634 as shown in FIG. 11. This allows the person to lie on a bed 632 while he is being exposed to the radiation. In the embodiment shown in FIG. 11, the apparatus 600 has a swiveling support for fixation on the wall 634 or the ceiling. The support is pivotable about spindle 638 to move apparatus 600 into a position which is convenient for use.

It is to be noted that the apparatus 600 of the embodiments shown in FIGS. 10 and 11 may be a single or dual-mode apparatus, e.g. for wrinkle reduction treatment alone or for wrinkle reduction treatment and acne treatment, depending on the selected mode of operation.

1. An apparatus for cosmetic skin rejuvenation treatment, comprising a continuous wave light source accommodating a high-intensity discharge lamp (104; 204; 304; 604), said high-intensity discharge lamp having at least one predominant spectral peak between 550 and 700 nm.

2. The apparatus of claim 1, wherein the high-intensity discharge lamp has a salt filling, said salt filling comprising at least one component from the group of lithium halide, sodium halide, cesium halide, calcium halide, or gallium halide.

3. The apparatus of claim 1, wherein the salt filling comprises at least one component from the group of lithium iodide, sodium iodide, cesium iodide, calcium iodide, or gallium iodide.

4. The apparatus of claim 1, wherein the high-intensity discharge lamp has a salt filling comprising lithium iodide and gallium iodide.

5. The apparatus of claim 1, wherein the high-intensity discharge lamp has a gas filling, said gas filling comprising at least one component from the group consisting of argon, xenon, krypton, and neon.

6. The apparatus of claim 1, wherein the high-intensity discharge lamp has a plasma chamber (424; 524), said plasma chamber having a quartz glass wall or a ceramic wall.

7. The apparatus of claim 1, wherein the continuous wave light source is adapted to operate with an alternative high-intensity discharge lamp for treatment of a skin disorder.

8. The apparatus of claim 7, wherein the alternative high-intensity discharge lamp (212; 312) has a predominant spectral peak between 390 and 430 nm.

9. The apparatus of claim 7, wherein the continuous wave light source has means (102) for releasably receiving the high-intensity discharge lamp or the alternative high-intensity discharge lamp.

10. The apparatus of claim 7, wherein the high-intensity discharge lamp has a first salt filling comprising lithium iodide, and the alternative high-intensity discharge lamp has a second salt filling comprising gallium iodide and/or aluminum iodide.

11. The apparatus of claim 7, wherein the continuous wave light source has means (214) for pivoting one of the high-intensity discharge lamp or the alternative high-intensity discharge lamp into a position suitable for use.

12. The apparatus of claim 7, wherein the continuous wave light source comprises said high-intensity discharge lamp (304) and said alternative high-intensity discharge lamp (312), and a control element (316) enabling a user to select either a first mode of operation, in which at least the high-intensity discharge lamp is switched on for skin rejuvenation treatment, or a second mode of operation, in which at least the alternative high-intensity discharge lamp is switched on for skin disorder treatment.

13. The apparatus of claim 1, wherein the apparatus is portable.

14. The apparatus of claim 1, wherein the apparatus is a table-top unit.

15. The apparatus of claim 1, wherein the apparatus is adapted to be hung on a wall or ceiling.

16. The apparatus of claim 1, wherein the apparatus is adapted to provide a radiation dose of at least 10 J/cm2 onto a skin area.

17. A lamp suitable for use in an apparatus according to claim 1.

18. Use of an apparatus in accordance with claim 1 for anti-wrinkle treatment of a person, wherein the lamp has a salt filling comprising lithium iodide.

19. Use of an apparatus in accordance with claim 1 for acne treatment of a person.

20. Use of a high-intensity discharge lamp (104; 204; 304; 604) for anti-wrinkle treatment of a person, the high-intensity discharge lamp having a salt filling comprising lithium iodide.
